(19) United States
(12) Patent Application Publication
(10) Pub. No.: US 2010/0241176 A1
(43) Pub. Date: Sep. 23, 2010

(54) FASTENING ELEMENT

(76) Inventor: Helke Lob, Munchen (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/734,000

(22) PCT Filed: Jul. 4, 2008
(86) PCT No.: PCT/EP2008/058728

§ 371 Date: Apr. 1, 2010

(30) Foreign Application Priority Data
Jul. 17, 2007 (DE) 10 2007 033 703.7

(51) Int. Cl. A61B 17/86

(52) U.S. Cl. 606/313

(57) ABSTRACT

A fastening element, particularly a peg, for fastening bone fragments or the like, is divided into at least two partial elements (10, 12) in the longitudinal direction. The partial elements encompass an expansion element (14). The partial elements and the expansion element being designed and arranged relative to each other such that the expansion element is located between the partial elements. A displacement of the expansion element in the longitudinal direction of the fastening element effecting a displacement of the partial elements in the lateral direction such that the partial elements are forced apart in the lateral direction and the fastening elements are brought from a closed position into an expanded position. The first partial element (10) having a protrusion (110) at the proximal longitudinal end of the inwardly directed sliding surface (42). The corresponding second partial element (12) at the proximal longitudinal end of the inwardly directed sliding surface (42) comprises a corresponding recess (120). The expansion element having a recess (14) at the proximal longitudinal end. The first and second partial elements and the expansion element are designed and arranged relative to each other such that, in the closed state of the fastening element and in the expanded state thereof, the protrusion (110) of the first partial element is introduced into the recess (120) of the second partial element at least partially.

### BACKGROUND OF THE INVENTION

1. Field of the Invention

The invention relates to a fastening element, in particular a screw anchor for fixating e.g. bone fragments, in particular spongiosa bone fragments, thus it is a fastener for surgical applications.

2. Description of the Background Art

The fastening element is divided in longitudinal direction into at least two partial elements, which are movable relative to one another based on their separation transversal to the longitudinal direction, thus in lateral direction. The partial elements enclose a spreader element. The spreader element and the partial elements are configured and disposed relative to one another, so that the spreader element is disposed between the partial elements and so that a movement of the spreader element in longitudinal direction of the fastening element causes a movement of the partial elements in lateral direction, so that the partial elements are driven apart from one another in lateral direction as a function of a movement of the spreader element, and the fastening element is thus moved from a closed state to a spread state.

A fastening element is of this type is known e.g. from WO 96/16607.

Such fastening elements are e.g. used like a screw anchor for fixating bone elements. Thus, the fastening element is inserted into a bore hole and subsequently spread through a spreader element. Due to the spreading, the fastening element is fixated at both bone fragments, which fixates the two bone elements relative to one another.

WO 2006/089929 describes a fastening element, which has a pull element actuated from the proximal end of the fastening element. The pull element is connected to the spreader element at least temporarily, so that actuating the pull element through pulling of the pull element causes a longitudinal movement of the spreader element and a spreading of the fastening element.

In view of this, there is a need to improve the handling of the fastening element. In particular, there is a need to provide a fastening element which can also be used in portions of the bone substance which have a soft consistency, like spongiosa or flat bones. Spongiosa is disposed on the inside of the bones and is enveloped on the outside by the kortikalis (substantia compacta). This is a spongy system of fine bone trabeculae, which form a softer but still stable bone structure by saving bone substance.

### SUMMARY OF THE INVENTION

According to the invention, the need is filled by a fastening element, in particular a screw anchor for fixating bone fragments or similar, which is separated into at least two partial elements in longitudinal direction, which jointly enclose a spreader element. The partial elements and the spreader element are configured and disposed relative to one another, so that the spreader element is disposed between the partial elements and a movement of the spreader element in longitudinal direction of the fastening element causes a movement of the partial elements in lateral direction, so that the partial elements are driven apart from one another in lateral direction, and the fastening elements is thus brought from a closed state into a spread state. The partial elements each have a respective inwardly oriented sliding surface, which corresponds to a respective surface of the spreader element and which extends in parallel to the respective surface in closed state. The fastening element has a distal longitudinal end and a proximal longitudinal end, and has to be inserted with its distal longitudinal end forward. The fastening element includes a pull element, which can be actuated from the proximal end of the fastening element, and which is connected to the spreader element at least temporarily, so that an actuation of the pull element by pulling at the pull element causes the longitudinal movement of the spreader element which causes the spreading of the fastening element. The spreader element does not protrude beyond the proximal end of the fastening element defined by the partial elements nor beyond the distal end of the fastening element defined by the partial elements in closed state of the fastening element or in its spread state.

Either at least one of the partial elements or the spreader element have a protrusion on its sliding surface facing the spreader element or the partial element, and the respective other element, thus the spreader element or the partial element, provides a longitudinal stop corresponding to the protrusion at which the protrusion contacts in longitudinal direction of the fastening element, when the fastening element has reached its maximum spread condition. The longitudinal stop can be e.g. the end of an elongated recess in a sliding surface disposed opposite to the protrusion.

In this way, the interaction of the protrusion and the longitudinal stop prevent the spreader element from being pulled in a proximal direction beyond the predetermined maximum point or out between the partial elements. Thus, the maximum possible spreading of the fastening element is predetermined.

The protrusion can be an integral component of one or plural partial elements or the spreader element. The protrusion can also be a separate component which is inserted into the respective partial element or the spreader element, which is preferably made of a bio resorbable plastic material like the partial elements or the spreader element themselves.

The protrusion can also be created by an offset in a respective partial element sliding surface. In this case, the longitudinal stop in the respective opposed sliding surface is also an offset, so that the sliding surfaces disposed opposite to one another comprise at least two partial sliding surfaces which are offset from one another and which can slide on one another since they are respectively disposed opposite to one another. This solution leads to a relatively simple geometry, but it can lead to the partial sliding surfaces sliding on top of one another becoming so small that the surface pressure acting between them becomes excessive.

Preferably, a first partial element has a protrusion at the proximal longitudinal end of the inwardly oriented sliding surface and a corresponding second partial element has a corresponding recess at the proximal longitudinal end of the inward oriented sliding surface and the spreader element has a recess at the proximal longitudinal end wherein the first and second partial element and the spreader element are configured and disposed relative to one another, so that the protrusion extends in the close state of the fastening element through the recess at least partially into the recess of the second partial element and the protrusion extends at least partially into the recess of the spreader element in the spread condition.

The invention is based on the finding that a fastening element according to the invention, in particular a screw anchor, is preferable over prior art screw elements, in particular for fixating spongiosa bone elements, this means bone elements which have a softer consistency.

It was found with fastening elements, in particular prior art screw anchors, that in particular bone material like spongiosa, when the fastening element is inserted and subsequently spread, does not put sufficient pressure on the fastening element in lateral direction, so that in the worst case, when the pull element is actuated by pulling into the proximal direction, the spreader element is pulled at least partially or entirely beyond the proximal end of the fastening element. Thus, the fastening action of the fastening element cannot be utilized sufficiently, so that the bone elements can grow together. Thus, it can happen in the worse case that the remaining fastening element has to be completely removed from the bone elements and a new fastening element has to be inserted.

Contrary thereto, the fastening element according to the invention has a first partial element which includes a protrusion at the proximal longitudinal end of the inwardly oriented sliding surface. This protrusion extends in closed state through the recess of the spreader element at least partially into the corresponding recess of a second partial element and in spread state, the protrusion of the first partial element extends at least partially into the recess of the spreader element and optionally into the recess of the second partial element. The recess of the spreader element is configured, so that the surface of the protrusion of the first partial element which is aligned transversally to the longitudinal side and to the distal end of the fastening element, is in contact with, thus “hits”, the surface of the spreader element at least in spread condition of the fastening element, which surface is aligned transversal to the longitudinal direction and to the distal end of the recess. This facilitates that, in spite of the low counter pressure of the spongiosa bone elements, the spreader element cannot be moved beyond the position of the stop of the protrusion at the spreader element when the pull elements are actuated in the proximal direction. This also facilitates an optimum fixation of the bone elements which shall grow together.

A fastening element according to the invention is preferable in particular when the protrusion of the first partial element extends in spread condition of the fastening element through the recess of the spreader element into the corresponding recess of the second partial element. This additionally prevents a movement of the partial elements relative to one another in longitudinal direction.

In a preferred embodiment the fastening element according to the invention is characterized in that the protrusion of the first partial element, the corresponding protrusion of the second partial element and the protrusion of the spreader element are aligned in parallel to the longitudinal extension of the fastening element and more preferably disposed additionally in the center. In the center, in the sense of the present invention, means that the protrusion, the recess and the cut out at the proximal end are disposed respectively centered with respect to the longitudinal direction of the first partial element, the second partial element and the spreader element.

The protrusion of the first partial element and the corresponding recess of the second partial element can have any three dimensional corresponding shapes which cause the protrusion to be in contact with the distal end of the recess of the spreader element in spread condition which prevents a movement of the spreader element beyond this point. This means e.g. cuboid shapes, in particular cubic shapes, pyramid shapes, cone shapes, etc. A cuboid shape is preferred in particular for the protrusion of the first partial element and the recess of the second partial element, wherein the protrusion has a volume, so that it can be inserted into the recess.

Preferably, the protrusion of the first partial element is made from the same material as the first partial element or the components of the fastening element.

In a preferred embodiment, the fastening element according to the invention is characterized in that the recess extends from the proximal end of the spreader element so far into the distal direction of the fastening element, so that in spread condition of the fastening element, a surface of the protrusion which is disposed transverse to the longitudinal direction and simultaneously in the direction of the distal end of the partial element, is in contact with a surface of the spreader element which is disposed transverse to the longitudinal direction and simultaneously disposed in the direction of the distal end of the recess of the spreader element and the spreader element does not protrude beyond the proximal end of the fastening element defined by the partial elements.

A spreader element which can be actuated through a pull element in a retro grade manner has several advantages. Thus e.g. spreading the spreader element can be provided without force components impacting the body of the patient in longitudinal direction of the fastening element during the process. This is possible since a compression force can be imparted upon the proximal end of the fastening element, which compression force corresponds to the pull force imparted upon the pull element, so that both forces compensate one another.

Furthermore, the pull element can be configured as a wire, a string, or also as a surgical thread. In the latter case, the already existing instruments which e.g. facilitate sewing up wounds can also be used for tightening and subsequent knotting of the pull element.

Thus the pull element is preferably configured as a thread, e.g. a surgical thread, a wire or a string.

As a matter of principle, the spreader element can have many configurations. Thus, it is possible to divide the fastening element along its longitudinal direction into four partial elements which are evenly distributed over the circumference of the fastening element. Each of these four partial elements can e.g. have a cross sectional shape which corresponds to the cross sectional shape of a quarter circle, which has a recess in its center which yields a rectangular square in combination with the recesses at the other three partial elements. This rectangular square can be advantageously configured, so that its diagonals extend in the direction of the half angle line of the quarter circle shaped partial element cross section of each respective partial element. The spreader element can then have a pyramid shape, wherein one of the respective edges extending to the tip of the pyramid extends through a groove provided by the recess in the cross section of the respective partial element.

The configuration of the partial elements and of the spreader elements is provided, so that the spreader element in the closed state of the fastening element and also in its spread state neither protrudes beyond the proximal end of the fastening element defined by the partial elements, nor beyond the distal element of the fastening element defined by the partial elements, but so that it is completely enclosed by both partial elements in any state of the fastening element.

Independent from this length sizing according to the invention it is an independent partial element feature of the invention to size the groove for the pull element, so that the pull element in any state of the fastening element is disposed between its partial elements and the spreader element, so it is freely moveable in longitudinal direction of the pull element and in parallel to the recess of the spreader element.

Preferably, the fastening element comprises two partial elements. In this case, the spreader element is preferably configured wedge shaped and tapers in proximal direction of the fastening element. The two partial elements then preferably have an inward oriented sliding surface which corresponds to a wedge surface of the spreader element and extends in closed condition of the fastening element in parallel to the respective partial element wedge surface, wherein the first partial element comprises a protrusion at the proximal end and the second partial element comprises a corresponding protrusion, and the wedge shaped partial element comprises a protrusion at the proximal end. This provides that the fastening element simultaneously spreads at the proximal end and at the distal end when the pull element is actuated. The preferred embodiment of the general configuration according to the invention can also be applied to a preferred a wedge shaped embodiment of the spreader element and the corresponding partial elements of the fastening element according to the invention.

In particular, the configuration of the spreader element as a relatively pointed and thus flat wedge facilitates the routing of a thread, string or wire shaped pull element about the distal end of the spreader element and the routing of the pull element from there along the two wedge surfaces, so that the two free ends of the pull element protrude beyond the proximal end of the fastening element and can be actuated from there.

In order to fixate the pull element with respect to its position, which pull element is routed around the wedge shaped spreader element, and in order to facilitate that the respective partial element wedge surface of the spread element can directly impact the respective sliding surface of the partial element, without the pull element preventing a direct contact and without the pull element being clamped in its longitudinal direction, preferably at least one groove for receiving the pull element is provided which extends on the outside of the spreader element in longitudinal direction of the fastening element and simultaneously in parallel to the recess of the spreader element. A respective groove in a wedge surface preferably extends along the distal end of the spreader element and then further in the other respective wedge surface. This yields a continuous circumferential groove. Preferably, two such grooves are provided, preferably both in parallel to the recess of the spreader element, so that the fastening element can also comprise two pull elements, e.g. configured as threads.

Instead of the spreader element comprising grooves, or in addition thereto, the grooves can also be provided on the sliding surfaces of the partial elements of the fastening element in those surfaces, where a respective partial element spreader element slides along, when the fastening element is being spread.

Respective grooves for the pull element or the pull elements are provided in any case thus independently from the shape of the spreader element are provided at the spreader element or at the partial elements to be spread of the fastening element, preferably sized, so that a respective pull element, e.g. a thread can be freely moved in non spread state and also in spread state of the fastening element. Namely this means that both thread ends proximally protruding from the spread fastening elements after its implementation can be knotted in an ideal manner, without the knotting being impaired by the fact that the thread is e.g. fixated by clamps. This way, also a complete removal of the pull element after expansion (spreading) of the fastening element is possible. The latter can be advantageous in particular when the wedge angle between the wedge surfaces of the pull element and the sliding surfaces of the partial element is sized, so that the spreader element is self hemming due to the friction forces acting between the wedge surfaces and the sliding surfaces, so that the spreader element after spreading does not slide back self acting, without any additional axial force introduction, into its initial position in non spread, closed state of the fastening element.

In closed state, the fastening element preferably has a cylindrical shape with circumferentially extending protrusions and recesses accordingly extending between the protrusions in circumferential direction. A respective cylindrical outer shape of the fastening element has the advantage that respective fitting round bore holes can be easily fabricated in bone fragments, which are to be fixated relative to one another. The protrusions extending in circumferential direction counteract a sliding of the fastening element in its longitudinal direction, while the fastening element is spread or after it has been spread.

Eventually, it is desirable that the partial elements of the fastening element cannot move relative to one another with respect to the longitudinal direction of the fastening element during the spreading of the fastening element or already during the insertion of the fastening element into a respective bore hole. In order to prevent such a relative longitudinal movement, it is provided that a separation surface between the partial elements along which separation surface the partial elements contact one another in closed condition of the fastening element, comprises a protrusion in circumferential direction. This discontinuity of the separation surface provides that at least one separation surface section is created which extends transversal to the longitudinal direction of the fastening element, which counteracts a movement of the partial elements in longitudinal direction relative to one another.

The sliding surface of each respective partial element is preferably the base of a recess which is recessed into the respective separation surface of the partial element. This recess preferably has a rectangular cross section. Accordingly, also the cross sections of the spreader element extending transverse to the longitudinal direction of the fastening element are rectangular. The two wedge surfaces of the spreader element are then adjacent at their longitudinal edges to longitudinal side surfaces of the spreader element which extend in parallel to one another. At its distal end, the spreader element is preferably disposed in a semi cylindrical shape and thus, so that the two wedge surfaces of the spreader element transition at their distal ends into the circumferential surface of the semi cylindrical shape. When, according to a preferred embodiment, the pull element is run about the distal end of the spreader element, the semi cylindrical shape has the advantage that the bending radius of the pull element can be kept as large as possible in this portion, so that there are no peak tensions which facilitate a tearing of the pull element. The semi cylindrical shape of the distal end of the spreader element furthermore supports the recited advantageous moveability of the pull element in its respective longitudinal direction.

Eventually the spreader element is advantageously sized in longitudinal direction, so that it does not protrude proximally from the fastening element even when the fastening element is spread to its maximum extent. The spreader element does not protrude from the distal end of the fastening element either when the spreader element is closed. In that state of the fastening element, the spreader element is preferably completely disposed within the fastening element.

Further advantageous embodiments of the spreader element can be derived from the patent claims and from the subsequent description.

With respect to the intended application of the fastening element, namely connecting bone fragments, preferably spongiosa bone fragments which shall be fixated relative to one another through the fastening element, so that they then grow together again in a natural healing process, it is particularly advantageous when the fastening element is resorbed by the body over time. Thus, the components of the fastening element are preferably made from a bio resorbable plastic material.

### BRIEF DESCRIPTION OF THE DRAWINGS

The invention will now be described in more detail with reference to an embodiment illustrated in several drawing figure, wherein:

FIG. 1 is a perspective view illustrating the fastening element according to the invention in a closed state;

FIG. 2 is a perspective view of the fastening element in a spread state;

FIG. 3 is a perspective view illustrating the first partial element and the second partial element and the spreader element of the fastening element according to the invention;

FIG. 4 is a perspective view illustrating the first partial element and the second partial element and the spreader element of the fastening element according to the invention;

FIG. 5 illustrates the first partial element of a fastening element according to the invention in a view of its longitudinal side;

FIG. 6 illustrates the first partial element of a fastening element according to the invention in a cross section view relative to the longitudinal direction;

FIG. 7 illustrates the first partial element of a fastening element according to the invention in a view of an outer surface of a fastening element;

FIG. 8 illustrates the first partial element of a fastening element according to the invention in a perspective view;

FIG. 9 illustrates the second partial element of a fastening element according to the invention in a view of a longitudinal side;

FIG. 10 illustrates the second partial element of a fastening element according to the invention in a cross section view relative to the longitudinal direction;

FIG. 11 illustrates the second partial element of a fastening element according to the invention in a view of the outer surface of the fastening element;

FIG. 12 illustrates the second partial element of a fastening element according to the invention in a perspective view;

FIG. 13 illustrates the spreader element of a fastening element according to the invention in a view of a longitudinal side;

FIG. 14 illustrates the spreader element of a fastening element according to the invention in a cross section view relative to the longitudinal direction;

FIG. 15 illustrates the spreader element of a fastening element according to the invention in a view of the surface of the spreader element, which is in contact with a sliding surface of a partial element of the fastening element; and

FIG. 16 illustrates the spreader element of a fastening element according to the invention in a perspective view.

### DETAILED DESCRIPTION OF THE INVENTION

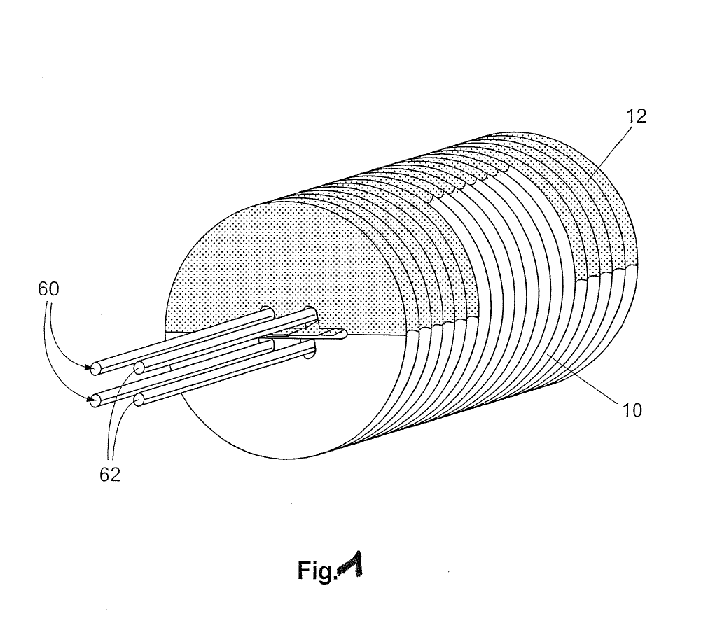

FIG. 1 illustrates the fastening element according to the invention in closed state with an inserted, but not visible spreader element 14. The spreader element 14 is enclosed between the two partial elements 10 and 12 over its entire length as also shown in FIG. 2 in spread condition. However, two respective partial element free ends 60 or 62 of two surgical threads being used as pull elements are visible. By pulling on the surgical threads being used e.g. as pull elements, the spreader element 14 is pulled deeper into the proximal direction of the fastening element and thus drives the two partial elements 10 and 12 laterally apart from one another, so that the spreader element is spread as illustrated in FIG. 2.

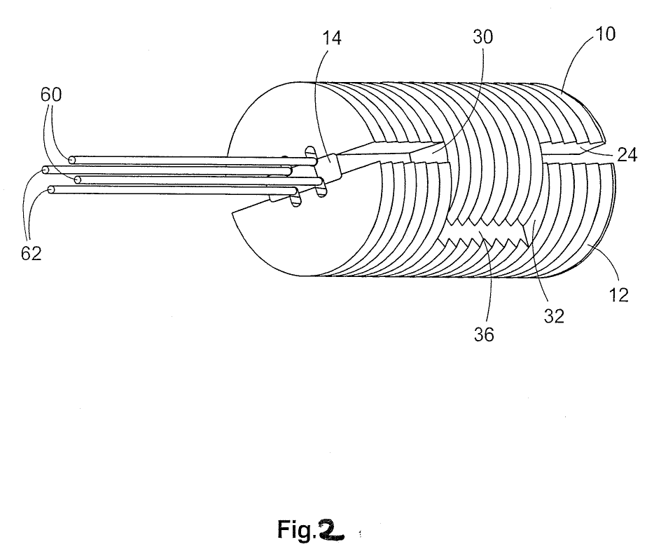

FIGS. 3 and 4 show components of a fastening element according to the invention, thus two partial elements 10 and 12 and a spreader element 14, wherein the pull element is not shown. As can be derived from FIG. 1, the two partial elements 10 and 12 can be assembled, so that the fastening element has a cylindrical outer shape. The two partial elements 10 and 12 then enclose a wedge shaped opening (not shown) between one another into which the spreader element 14 fits precisely.

The wedge shaped opening is created by the recesses 20, 22 in the respective partial element 10 or 12. The recesses 20 or 22 are disposed on the inside of a partial element 10 or 12 and they are recesses with respect to a separation surface which extends substantially parallel to the longitudinal direction of the fastening element. The separation surface leads to partial separation surfaces 24 or 26 at the partial elements 10 or 12, which are disposed on top of one another in the closed state of the fastening element. In a center longitudinal section of the fastening element, the separation surface has a discontinuity, so that partial separation surface sections 30, 32 which extend transversal to the longitudinal direction of the fastening element are created at the partial elements 10 or 12. These partial surface sections 30 and 32 provide that the partial elements 10 and 12 cannot be moved longitudinally relative to one another in closed and also in spread condition of the fastening element. Partial surface sections 34 and 36 which are parallel to the longitudinal axis furthermore prevent the partial elements 10 and 12 from moving laterally relative to one another.

As can be derived particularly well from FIGS. 3 and 4, the respective recesses in the partial elements 10 or 12 respectively have a base which is used as a sliding surface 40 or 42. When the spreader element 14 is inserted, these sliding surfaces 40 or 42 interact with respective partial element wedge surfaces 44 or 46 of the spreader element 14. The wedge surfaces 44 and 46 of the spreader element 14 transition at their distal end into a semi cylindrical shape of the spreader element 14. Two grooves 50 and 52 extend respectively preferably in longitudinal direction from a wedge surface over the semi cylindrical shape to the other wedge surface. The grooves 50 or 52 are sized, so that they completely receive a thread, which is used as a pull element, so that a longitudinal movement of the thread between the sliding surfaces 40 and 42 and the respective opposed wedge surfaces 40 and 46 is not impeded.

It can be furthermore derived from FIG. 3 that the sliding surface 40 or the first

partial element 10 has a protrusion 110 at the proximal end of the partial element 10.

This protrusion 110 corresponds to the recess 120 in the sliding surface 42 of the second partial element 12 which is shown in particular in FIG. 4. The spreader element 14 comprises a recess 140 which is configured, so that the protrusion 110 of the first partial element 10 extends in closed state of the fastening element into this recess 140 and at least partially into the recess 120. In spread state of the fastening element, the protrusion 110 extends at least partially into the recess 140 and optionally into the recess 120. Preferably, the surface 111 (FIG. 5) of the protrusion of the first partial element 10 which is disposed transversely to the longitudinal direction and in the direction of the distal end of the first partial element 10, is in contact in spread condition of the fastening element with the surface 141 of the spreader element, which is disposed transversely to the longitudinal direction and in the direction of the distal end of the spreader element. Through cooperation of the protrusion 110 of the first partial element 10 with the recess 140 of the spreader 14 that the spreader 14 is prevented from being pulled beyond the proximal end of the fastening element when sufficient pressure is applied to the pull elements 60 and 62 and/or little opposite pressure is applied by the bone material, in particular the spongiosa material. Thus, an optimum connection of the bone elements being connected is achieved particularly preferably in soft bone materials, like e.g. spongiosa.

When the protrusion 110 of the first partial element 10 extends also in spread condition at least partially into the recess 120 of the second partial element 12, this prevents a longitudinal movement and possibly a transversal movement of the partial elements 10 and 12, in particular when the pull elements 60 and 62 are actuated.

The wedge surfaces and the semi cylindrical shape transition laterally into the longitudinal side surfaces 54 of the spreader element 14. The edges between the wedge surfaces or the semi cylindrical shape and the longitudinal side surfaces 54 of the spreader element are rounded.

The grooves 50 and 52 are used for receiving a respective surgical thread as pull element. This becomes evident when viewing the FIGS. 1 and 2.

As can be derived in particular from the FIGS. 3 and 4, short groove section 70 are provided at the proximal end of the sliding surfaces in the partial elements 10 and 12, which groove sections correspond with respect to their positions with the grooves 52 in the spreader element 14.

FIG. 5 illustrates a view of the longitudinal side of the first partial element 10 with a protrusion 110. The disposition of the surface 111 of the protrusion which is in contact with the surface 141 of the spreader element is indicated. Furthermore, the recess 20 with the sliding surface 40 of the first partial element is shown.

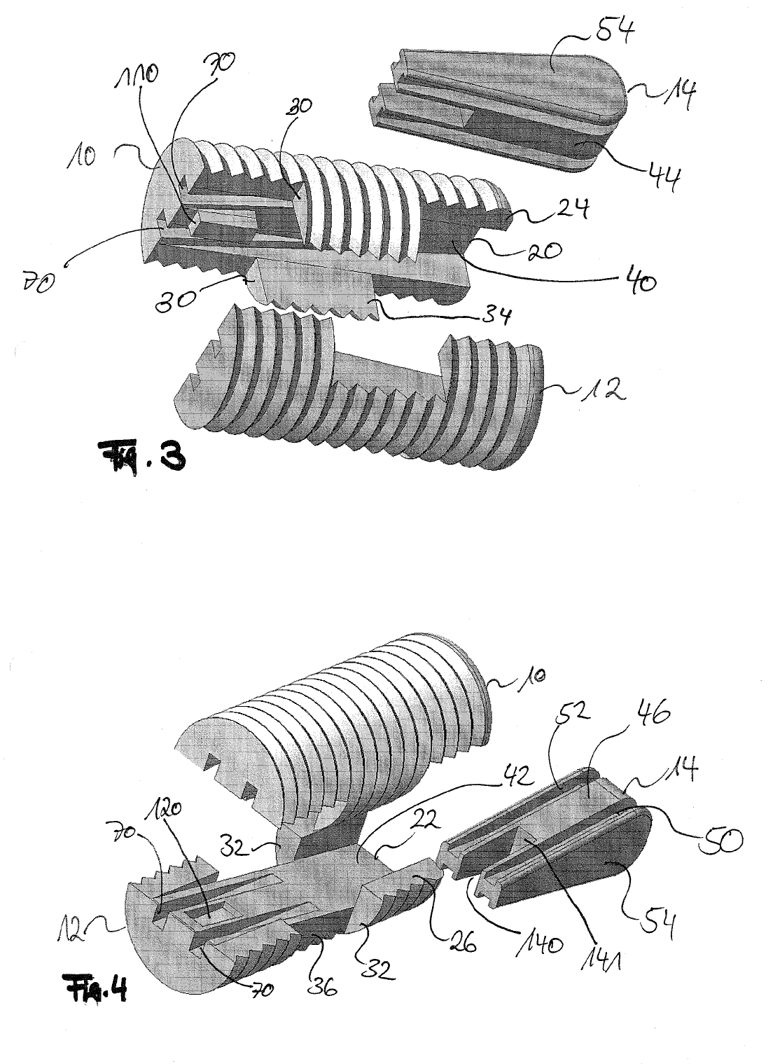

FIG. 6 illustrates the cross section of the first partial element 10 with the protrusion 110. Herein in particular the two recesses for the grooves 70 and the surfaces 30 and 34 of the first partial element are described as shown supra.

FIG. 7 illustrates a view of the outer surface (envelope) of the first partial element 10. Herein the serration of the enveloping surface is visible. Furthermore recesses for the grooves 70 can be derived from the dashed lines and also the location of the protrusion 110 can be derived.

FIG. 8 illustrates a perspective view of the first partial element 10, in particular the grooves 70 and the surface 30 are illustrated.

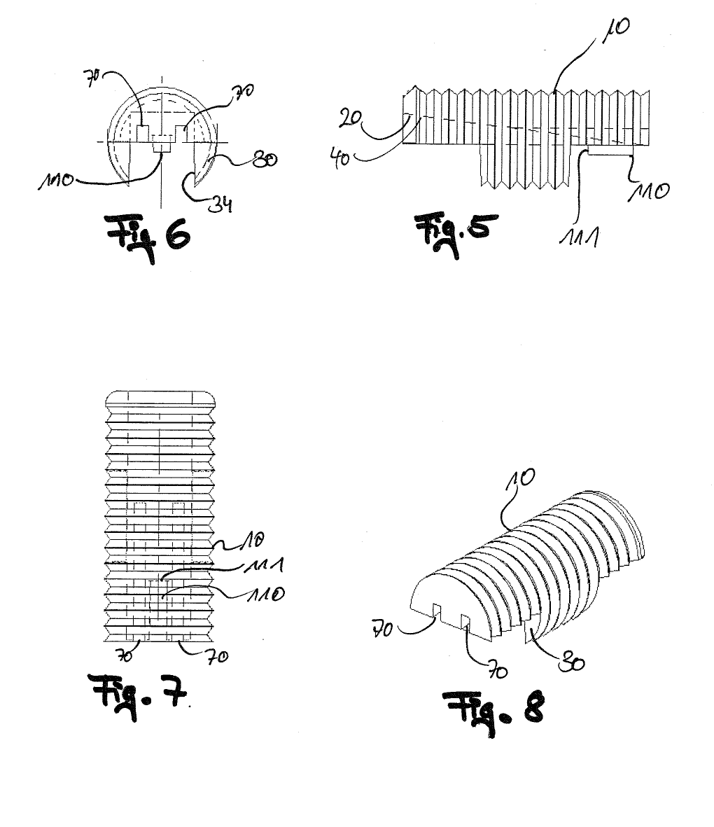

FIG. 9 illustrates a view of the longitudinal side of the second partial element 12 with a recess 120 (dashed).

FIG. 10 illustrates the cross section of the second partial element 12 with a recess 120 (dashed). Herein in particular the two recesses for the grooves 70 are illustrated.

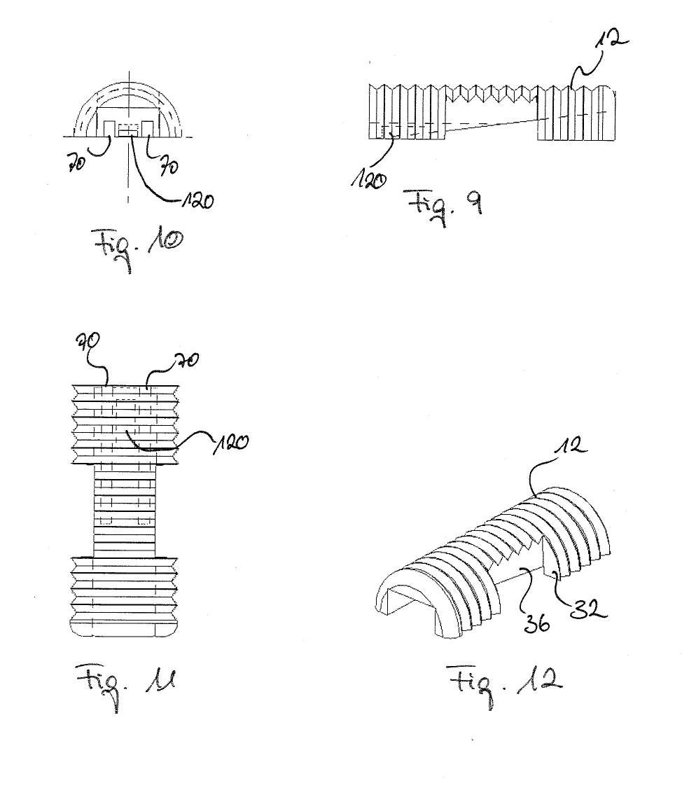

FIG. 11 illustrates a view of the outer surface (enveloping surface) of the second partial element 12. This illustrates the serration of the enveloping surface. Furthermore recesses for the grooves 70 are apparent from the dashed lines and also the location of the recess 120 is visible.

FIG. 12 illustrates the respective partial element view of the second partial element 12. In particular the grooves 70 and the surfaces 32 and 36 are depicted.

FIG. 13 illustrates a view of the longitudinal side of the spreader element of a fastening element according to the invention, wherein the surface of the longitudinal side 54 is illustrated. Additionally, the recess for the groove 50 is illustrated in dashed lines.

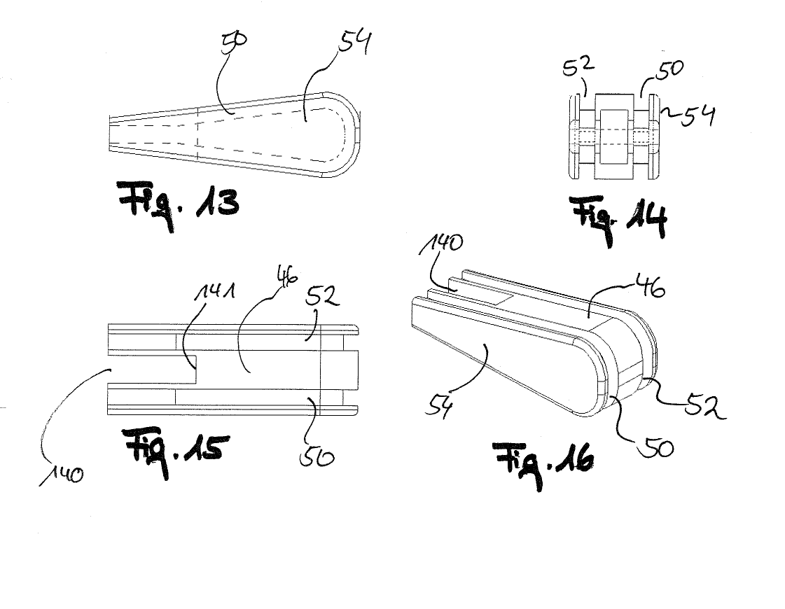

FIG. 14 illustrates the spreader element 14 of a fastening element according to the invention in cross section relative to the longitudinal direction, wherein in particular the grooves 50 and 52 and the location of the longitudinal surface 54 are depicted.

FIG. 15 illustrates the spreader element 14 of a fastening element according to the invention in a view of the surface 46 of the spreader element, which surface contacts the sliding surface 40 or 42 of the first or second partial element 10 or 12 of the fastening element. Additionally, the recess 140 and the surface 141 are located in contact the surface 111 of the protrusion of the first partial element preferably in spread condition. Furthermore, the recesses for the grooves 50 and 52 are depicted.

FIG. 16 illustrates the spreader element 14 of a fastening element according to the invention in a perspective view. Thus, in particular, the surface 46 which contacts the sliding surface 40 or 42 of the first or second partial element 10 or 12 of the fastening element, the surface 54 of the longitudinal side, the recesses for the grooves 50 and 52 and the recesses 140 of the spreader 14 are depicted.

As can be derived in particular in FIGS. 1-5, 7-9 and 11 and 12, the fastening element is serrated at its enveloping surface. This serration is caused by a plurality of circumferentially extending protrusions which are used for anchoring the fastening element in longitudinal direction.

1-23. (canceled)

24. A fastening element, in particular a screw anchor for fixating bone fragments or the like, comprising:
at least two partial elements dividing the fastening element in longitudinal direction and being laterally moveable relative to each other between closed and open states of the fastening element and thereby defining a variable opening for enclosing a spreader element, each of the at least two partial elements having an inwardly oriented sliding surface which respectively corresponds to a respective sliding surface of the spreader element and extends in closed condition parallel to its respective sliding surface of the spreader element, the at least two partial elements defining proximal and distal ends of the fastening element;
a spreader element having a distal longitudinal end and a proximal longitudinal end and inserted with the distal longitudinal end forward in the variable opening defined by the at least two partial elements without protruding beyond the proximal end of the fastening element defined by the at least two partial elements nor beyond the distal end of the fastening element defined by the partial elements in both the closed and open states of the fastening element, wherein a first one of the at least two partial elements has a protrusion orientated inward with respect to its inwardly oriented sliding surface and the spreader element has a corresponding longitudinal stop which the protrusion contacts when the spreader element has reached its most proximal relative position with respect to the first partial element and thus has reached its maximum spread state or the spreader element has a protrusion in one of its sliding surfaces and the corresponding sliding surface of at least one of the at least two partial elements comprises a corresponding longitudinal stop which the protrusion contacts when the spreader element has reached its most proximal relative position with respect to the at least two partial elements; and
a pull element at least temporarily connected with the spreader element, the pull element being actuatable from the proximal end of the fastening element so that actuation of the pull element by pulling at the pull element causes the longitudinal movement of the spreader element which causes the spreading of the fastening element;
whereby the at least two partial elements and the spreader element are configured and disposed relative to one another, so that the spreader element is disposed between the at least two partial elements so movement of the spreader element in a longitudinal direction of the fastening element causes a movement of the at least two partial elements in the lateral direction, so that the at least two partial elements are driven apart in lateral direction and the fastening element is thus moved from the closed state to the spread state.

25. A fastening element according to claim 24, wherein the protrusion disposed on the inwardly oriented partial surface of the first one of the at least two partial elements corresponds to a corresponding recess at the proximal longitudinal end of the inwardly oriented sliding surface of a second one of the at least two partial elements so that the protrusion is extendable at least partially into the recess in the closed state of the fastening element and the spreader element has a recess at the proximal longitudinal end forming the longitudinal stop so that the protrusion contacts a longitudinal end of the recess of the spreader element in maximum spread condition of the fastening element.

26. A fastening element according to claim 25, wherein the protrusion of the first one of the at least two partial elements, the corresponding recess of the second one of the at least two partial elements and the recess of the spreader element are longitudinally extended parallel to the longitudinal direction of the fastening element.

27. A fastening element according to claim 25, wherein the protrusion of the first one of the at least two partial elements, the corresponding recess of the second one of the at least two partial elements and the recess of the spreader element are centrally disposed with respect to a lateral extension of the fastening element.

28. A fastening element according to claim 25, wherein the protrusion of the first one of the at least two partial elements and the corresponding recess of the second one of the at least two partial elements respectively have a rectangular longitudinal cross section, wherein the length and the width of the protrusion corresponds to the length and width of the recess.

29. A fastening element according to claim 25, wherein the recess in the spreader element extends, starting at the proximal end of the spreader element, so far into the distal direction of the fastening element that, in maximum spread condition of the fastening element, a surface of the protrusion which is disposed transverse to the longitudinal direction and simultaneously in the direction of the distal end in the partial element, is in contact with a surface of the spreader element which is oriented transverse to the longitudinal direction and simultaneously in the direction of the distal end of the spreader element and the spreader element does not protrude beyond the proximal end of the fastening element defined by the at least two partial elements.

30. A fastening element according to claim 25, wherein the pull element is a thread, a wire, a string or similar.

31. A fastening element according to claim 30, wherein the pull element is run in a loop about the distal end of the spreader element, so that both free ends of the pull element are disposed at the proximal end of the fastening element.

32. A fastening element according to claim 30, wherein the spreader element has at least one groove extending in the longitudinal direction of the fastening element on the surfaces which correspond to the sliding surfaces of the at least two partial elements for receiving the pull element.

33. A fastening element according to claim 32, wherein the at least one groove is disposed in parallel to the recess of the spreader element.

34. A fastening element according to claim 32, wherein the at least one groove extends along both surfaces and is run around the distal end of the spreader element.

35. A fastening element according to claim 32, wherein the at least one groove in the spreader element comprises two grooves extending in parallel to one another and to the recess of the spreader element.

36. A fastening element according to claim 32, wherein the at least one groove is sized to completely receive the pull element, so that it can move freely therein in the longitudinal direction in any state of the fastening element.

37. A fastening element according to claim 24, wherein the spreader element is wedge shaped and tapers in the proximal direction of the fastening element.

38. A fastening element according to claim 37, wherein the spreader element has two wedge surfaces adjacent to longitudinal side surfaces of the spreader element, which side surfaces extending parallel to one another.

39. A fastening element according to claim 38, wherein the spreader element is rounded in a semi cylindrical shape at its distal end thereby defining a circumferential surface which respectively transitions into the wedge surfaces.

40. A fastening element according to claim 39, wherein face surfaces of the semi cylindrical shape respectively form a distal section of the longitudinal side surfaces of the spreader element.

41. A fastening element according to claim 39, wherein at least one groove is formed in the semi cylindrical shape, which at least one groove respectively extends into at least one corresponding groove in the wedge surfaces.

42. A fastening element according to claim 24, wherein the fastening element in its closed state is shaped like a cylinder with serrations in circumferential direction on an outer surface thereof.

43. A fastening element according to claim 24, wherein the at least two partial elements are two partial elements separated from one another along a separation surface and the separation surface has a discontinuity in longitudinal direction of the fastening element, so that at least one separation surface section is created which extends transversely to the longitudinal direction of the fastening element, which separation surface section counteracts movement of the partial elements relative to one another with reference to the longitudinal direction of the fastening element and which counteracts the spreading of the fastening element.

44. A fastening element according to claim 43, wherein the sliding surface of each respective partial element is a base wall of a recess formed in the respective separation surface of the partial element.

45. A fastening element according claim 24, wherein the cross sections of the spreader element extending transversely to the longitudinal direction of the fastening element respectively has a rectangular shape.

46. A fastening element according claim 24, wherein the at least two partial elements and the spreader element are made from bio resorbable plastic material.
